(19) United States
(12) Patent Application Publication
(10) Pub. No.: US 2009/0171093 A1
(43) Pub. Date: Jul. 2, 2009

(54) 3'-Fluoro-5'-hydroxythalidomide and derivatives thereof

(76) Inventors: Yoshio Takeuchi, Imizu (JP); Tomoya Fujiwara, Toyama (JP); Tomoyuki Saito, Sendai (JP)

Correspondence Address: WENDEROTH, LIND & PONACK, L.L.P., 1030 15th Street, N.W.,, Suite 400 East, Washington, DC, 20005-1503, US

(21) Appl. No.: 12/216,026

(22) Filed: Jun. 27, 2008

(30) Foreign Application Priority Data
Dec. 28, 2007 (JP) 2007-338439

(51) Int. Cl. C07D 401/04

(52) U.S. Cl. 546/200

(57) ABSTRACT

There are provided a novel 3′-fluoro-5′-hydroxythalidomide derivative and an optically active substance thereof A 3′-fluoro-5′-hydroxythalidomide derivative represented by the general formula [A]:

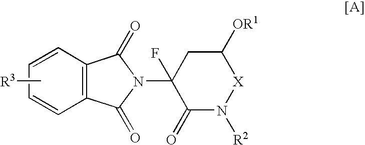

wherein R¹, R² and R³ are each a hydrogen atom; and X is C═O, and an optically active substance thereof. As analogs of thalidomide, the 3′-fluoro-5′-hydroxythalidomide derivatives are useful in the treatment of erythema nodosum of Hansen's disease, prurigo, rheumatism, Crohn's disease, graft-versus-host disease, Behcet's disease, myeloma, aphtha ulcer and the like. Further, the 3′-fluoro-5′-hydroxythalidomide derivative is useful as a drug used for treatment of multiple myeloma, TNF-α related diseases and the like.

### The present invention relates to novel 3′-fluoro-5′-hydroxythalidomide and derivatives thereof, and optically active substances thereof.

### BACKGROUND OF THE INVENTION

Thalidomide was put on the market as a sedative-hypnotic drug in 1958. However, it was revealed to be teratogenic to fetuses, and after 5 years from the release, it was withdrawn from the market. Thereafter, it has been revealed that thalidomide is effective for erythema nodosum of Hansen's disease, and since then, the effectiveness for various intractable diseases such as prurigo, rheumatism, Crohn's disease, graft-versus-host disease, Behcet's disease, myeloma and aphtha ulcer has been then reported. U.S. Food and Drug Administration (FDA) approved thalidomide as a therapeutic agent for erythema nodosum of Hansen's disease, and led to the approval for medication for multiple myeloma in combination with dexamethasone in May, 2006.

Although thalidomide has a chiral center and is sold as a racemic mixture, it is suggested that its teratogenicity is derived from the S-isomer and that the pure R-isomer could be used to avoid the teratogenicity (Arzneim. Forsch./Drug Res., Vol. 29, No. 10, pp 1640-1642, 1979). However, the R-isomer of thalidomide is easily epimerized under physiological conditions to produce the S-isomer (Biochem. Biophys. Res. Commun., Vol. 199, No.2, pp 455-460, 1994), and therefore it is expected for the detailed study whether or not harmful effects of this drug can be avoided (Lancet., Vol. 339, No. 8789, pp 365, 1992). Further, for the purpose of avoiding epimerization under physiological conditions, a derivative in which hydrogen at the 3-position of thalidomide is replaced with a fluorine atom has been proposed (Japanese Patent Laid-Open No. 2000-159761).

Although the mechanism for a wide variety of biological activities of thalidomide is unclear, the possibility is suggested that the actual active substance is thalidomide itself or the metabolites thereof (Current Drug Metabolism, Vol. 7, No. 6, pp 677-685, 2006). Further, as the metabolic pathways of thalidomide, the hydrolysis of thalidomide and the hydroxylation of thalidomide by liver have been proposed (Current Drug Metabolism, Vol. 7, No. 6, pp 677-685, 2006 and Biochem. Pharm., Vol. 55, No. 11, pp 1827-1834, 1998), and 5′-hydroxythalidomide has been especially reported as a hydroxylated metabolite (Anal. Chem., Vol. 74, No. 15, pp 3726-3735, 2002). However, it cannot be denied that even 5′-hydroxythalidomide may be epimerized in vivo, and the precise difference in biological activities between individual enantiomers cannot be discussed. In order to solve this problem, it is essential to develop a structurally similar, yet non-epimerizable analog corresponding to 5′-hydroxythalidomide.

### BRIEF SUMMARY OF THE INVENTION

The present inventors have succeeded in that the mixture of four stereoisomers of 3′-fluoro-5′-hydroxythalidomide is synthesized to prevent epimerization of 5′-hydroxythalidomide, and the mixture is separated to obtain each stereoisomer of 3′-fluoro-5′-hydroxythalidomide in optically pure form, leading to the completion of the present invention. Hereinafter, the present invention will be specifically described.

### BRIEF DESCRIPTION OF THE DRAWINGS

FIG. 1 depicts general formula [A].

FIG. 2 depicts general formula [B].

FIG. 3 depicts the synthesis of 3′-fluoro-5′-hydroxythalidomide starting from (5-hydroxy-2-oxopiperidin-3-yl)carbomic acid tertbutyl ester.

FIG. 4 depicts the synthesis of compound 7 from compound 5, and the synthesis of compound 9 from compound 7. FIG. 4 also depicts the synthesis of compound 8 from compound 6, and the synthesis of compound 10 from compound 8.

FIG. 5 depicts the optical resolution of compounds 9 and 10, and the general procedure of synthesizing optically pure compounds 11 and 12 from compounds 9 and 10, respectively.

FIG. 6 depicts the determination of absolute configuration of 3′-fluoro-5′-hydroxythalidomide.

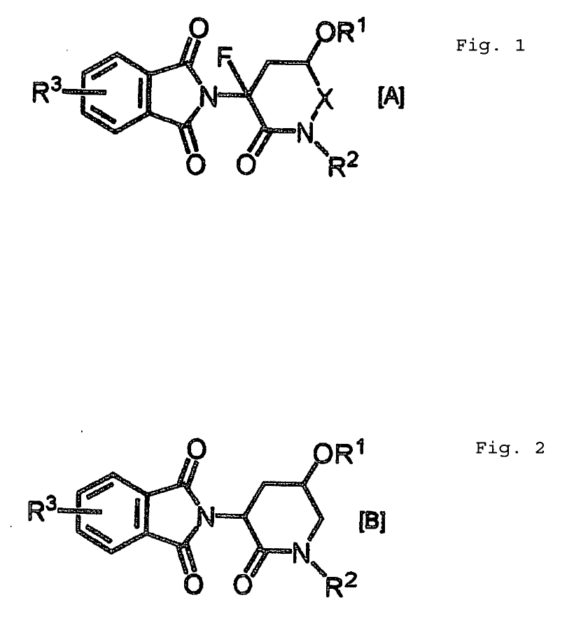

### DETAILED DESCRIPTION OF THE INVENTION

In the present specification, unless otherwise specified, each term has the following meanings. The term “halogen atom” means a fluorine atom, a chlorine atom, a bromine atom or an iodine atom; the term “alkyl group” means a linear or branched C₁₋₁₂ alkyl group such as a methyl, ethyl, propyl, isopropyl, butyl, isobutyl, tert-butyl, pentyl or hexyl group; the term “lower alkyl group” means a linear or branched C₁₋₆ alkyl group such as a methyl, ethyl, propyl, isopropyl, butyl, isobutyl, tert-butyl, pentyl or hexyl group; the term “lower alkenyl group” means a C₂₋₆ alkenyl group such as a vinyl, propenyl, butenyl, pentenyl or hexenyl group; the term “lower alkynyl group” means a C₂₋₆ alkynyl group such as an ethynyl, 2-propynyl or 2-butynyl group; the term “cycloalkyl group” means a cyclopropyl, cyclobutyl, cyclopentyl or cyclohexyl group; the term “alkoxy group” means a linear or branched C₁₋₁₂ alkyloxy group such as a methoxy, ethoxy, propoxy, isopropoxy, butoxy, isobutoxy, tert-butoxy, pentyloxy, hexyloxy, heptyloxy or octyloxy group; the term “lower alkoxy group” means a linear or branched C₁₋₆ alkyloxy group such as a methoxy, ethoxy, propoxy, isopropoxy, butoxy, isobutoxy, tert-butoxy, pentyloxy or hexyloxy group; the term “lower alkylthio group” means a linear or branched C₁₋₆ alkyl-S group such as a methylthio, ethylthio, propylthio, isopropylthio, butylthio or hexylthio group; the term “lower alkoxycarbonyl group” means a linear or branched C₁₋₆ alkyl-O—CO group such as a methoxycarbonyl, ethoxycarbonyl, propoxycarbonyl, isopropoxycarbonyl, butoxycarbonyl, tert-butoxycarbonyl or hexyloxycarbonyl group; the term “aryl group” means a phenyl, naphthyl, indanyl or indenyl group; the term “aralkyl group” means an aryl-C₁₋₆ alkyl group such as a benzyl, diphenylmethyl, trityl or phenethyl group; the term “acyl group” means a formyl group, an alkylcarbonyl group or an aroyl group; the term “alkylcarbonyl group” means a C₂₋₆ alkylcarbonyl group such as an acetyl or propionyl; the term “aroyl group” means an arylcarbonyl group such as a benzoyl or naphthylcarbonyl group; the term “acyloxy group” means an acyloxy group such as an acetyloxy, pivaloyloxy, phenylacetyloxy, 2-amino-3-methylbutanoyloxy, ethoxycarbonyloxy, benzoyloxy or 3-pyridylcarbonyloxy; the term “silyl group” means a tert-butyldimethylsilyl, trimethylsilyl, triethylsilyl, triisopropylsilyl or tert-butyldiphenylsilyl; the term “alkylsulfonate” means a C₁₋₆ alkylsulfonyl such as trifluoromethanesulfonyl or methanesulfonyl; and the term “arylsulfonate” means a benzenesulfonyl or 4-toluenesulfonyl.

A substituted group represented by R¹ includes a protecting group of a hydroxyl group which includes all groups which can be used as a usual protecting group of a hydroxyl group, and examples thereof include acyl groups such as benzyloxycarbonyl, 4-nitrobenzyloxycarbonyl, 4-bromobenzyloxycarbonyl, 4-methoxybenzyloxycarbonyl, 3,4-dimethoxybenzyloxycarbonyl, methoxycarbonyl, ethoxycarbonyl, tert-butoxycarbonyl, 1,1-dimethylpropoxycarnobyl, isopropoxycarbonyl, isobutyloxycarbonyl, diphenylmethoxycarbonyl, 2,2,2-trichloroethoxycarbonyl, 2,2,2-tribromoethoxycarbonyl, 2-(trimethylsilyl)ethoxycarbonyl, 2-(phenylsulfonyl)ethoxycarbonyl, 2-(triphenylphosphonio)ethoxycarbonyl, 2-furfuryloxycarbonyl, 1-adamanthylocycarbonyl, vinyloxycarbonyl, allyloxycarbonyl, S- benzylthiocarbonyl, 4-ethoxy-1-naphthyloxycarbonyl, 8-quinolyloxycarbonyl, acetyl, formyl, chloroacetyl, dichloroacetyl, trichloroacetyl, trifluoroacetyl, methoxyacetyl, phenoxyacetyl, pivaloyl and benzoyl; lower alkyl groups such as methyl, tert-butyl, 2,2,2-trichloroethyl and 2-trimethylsilylethyl; lower alkenyl groups such as allyl; aryl-lower alkyl groups such as benzyl, p-methoxybenzyl, 3,4-dimethoxybenzyl, diphenylmethyl and trityl; oxygen-containing and sulfur-containing heterocyclic groups such as tetrahydrofuryl, tetrahydropyranyl and tetrahydrothiopyranyl; lower alkoxy- and lower alkylthio-lower alkyl groups such as methoxymethyl, methylthiomethyl, benzyloxymethyl, 2-methoxyethoxymethyl, 2,2,2-trichloroethoxymethyl, 2-(trimethylsilyl)ethoxymethyl, 1-ethoxyethyl and 1-methyl-1-methoxyethyl; lower alkyl- and aryl-sulfonyl groups such as methansulfonyl and p-toluenesulfonyl; and substituted silyl groups such as trimethylsilyl, triethylsilyl, triisopropylsilyl, diethylisopropylsilyl, tert-butyldimethylsilyl, tert-butyldiphenylsilyl, diphenylmethylsilyl and tert-butylmethoxyphenylsilyl.

A substituted group represented by R² includes a protecting group of an amino group which includes all groups which can be used as a usual amino protecting group, and examples thereof include acyl groups such as trichloroethoxycarbonyl, tribromoethoxycarbonyl, benzyloxycarbonyl, p-nitrobenzyloxycarbonyl, o-bromobenzyloxycarbonyl, (mono-, di-, tri-)chloroacetyl, trifluoroacetyl, phenylacetyl, formyl, acetyl, benzoyl, tert-amyloxycarbonyl, tert-butoxycarbonyl, p-methoxybenzyloxycarbonyl, 3,4-dimethoxybenzyloxycarbonyl, 4-(phenylazo)benzyloxycarbonyl, 2-furfuryloxycarbonyl, diphenylmethoxycarbonyl, 1,1-dimethylpropoxycarbonyl, isopropoxycarbonyl, phthaloyl, succinyl, alanyl, leucyl, 1-adamantyloxycarbonyl and 8-quinolyloxycarbonyl; aryl-lower alkyl groups such as benzyl, diphenylmethyl and trityl; arylthio groups such as 2-nitrophenylthio and 2,4-dinitrophenylthio; alkyl- or aryl-sulfonyl groups such as methanesulfonyl and p-toluenesulfonyl; di-lower alkylamino-lower alkylidene groups such as N,N-dimethylaminomethylene; aryl-lower alkylidene groups such as benzylidene, 2-hydroxybenzylidene, 2-hydroxy-5-chlorobenzylidene and 2-hydroxy-1-naphthylmethylene; nitrogen-containing heterocyclic alkylidene groups such as 3-hydroxy-4-pyridylmethylene; cycloalkylidene groups such as cyclohexylidene, 2-ethoxycarbonylcyclohexylidene, 2-ethoxycarbonylcyclopentylidene, 2-acetylcyclohexylidene, and 3,3-dimethyl-5-oxycyclohexylidene; diaryl- or diaryl-lower alkylphosphoryl groups such as diphenylphosphoryl and dibenzylphosphoryl; oxygen-containing heterocyclic alkyl groups such as 5- methyl-2-oxo-2H-1,3-dioxol-4-yl-methyl; and substituted silyl groups such as trimethylsilyl.

The present invention provides 3′-fluoro-5′-hydroxythalidomide derivatives represented by the following general formula [A]:

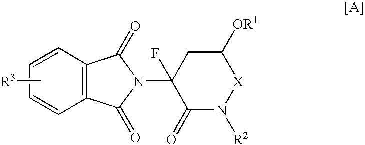

[wherein R¹ represents a hydrogen atom, or an optionally substituted silyl, alkyl, aryl or acyl group; R² represents a hydrogen atom, or an optionally substituted hydroxyl, alkoxy, acyl, acyloxy or alkyl group; R3 represents 1 to 4 the same or different atoms or groups selected from a hydrogen atom, a lower alkyl group, a lower alkoxy group, a halogen atom, a halogenated lower alkyl group, an amino group which may have a substituent, a lower alkylthio group, a lower alkoxycarbonyl group, a carbamoyl group which may have a substituent, a cyano group, a lower alkenyl group and a lower alkynyl group; and X represents CH₂ or C═O], and a (3′S, 5′R)-3′-fluoro-5′-hydroxythalidomide derivative, a (3′R, 5′S)-3′-fluoro-5′-hydroxythalidomide derivative, a (3′S, 5′S)-3′-fluoro-5′-hydroxythalidomide derivative, and a (3′R, 5′R)-3′-fluoro-5′-hydroxythalidomide derivative in optically active forms.

The optically active substance usually contains pure optical isomer in a range from 75% to 100%, preferably from 85% to 100% and most preferably from 95% to 100%.

A 3′-fluoro-5′-hydroxythalidomide derivative in which R¹ is a hydrogen atom or an optionally substituted silyl group; R² is a hydrogen atom or an optionally substituted acyl group; and R3 is a hydrogen atom in the general formula [A], and an optically active substance thereof are preferred, and a 3′-fluoro-5′-hydroxythalidomide derivative in which R² and R3 are each a hydrogen atom and X is C═O in the general formula [A], and an optically active substance thereof are more preferred.

In addition, R¹ may be a hydroxyl protecting group and R² may be an amino protecting group.

The compound of the general formula [A] can be produced, for example, by the following method.

The compound of the general formula [A] can be produced by treating 3-phthalimidopiperidin-2-one derivatives represented by the general formula [B]:

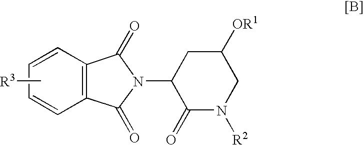

[wherein R¹ represents a hydrogen atom, or an optionally substituted silyl, alkyl, aryl or acyl group; R² represents a hydrogen atom, or an optionally substituted hydroxyl, alkoxy, acyl, acyloxy or alkyl group; and R3 represents 1 to 4 the same or different atoms or groups selected from a hydrogen atom, a lower alkyl group, a lower alkoxy group, a halogen atom, a halogenated lower alkyl group, an amino group which may have a substituent, a lower alkylthio group, a lower alkoxycarbonyl group, a carbamoyl group which may have a substituent, a cyano group, a lower alkenyl group or a lower alkynyl group], with a base and an electrophilic fluorinating reagent, and then subjecting a compound in which R² is a protecting group of an amino group to deprotection of an amino group as required, or further subjecting the resulting derivative to treatment with an oxidizing agent or subjecting a compound in which R¹ is a silyl group to removal of a silyl group.

Examples of the electrophilic fluorinating reagent used in this synthetic procedure include N-fluorobenzenesulfonimide, molecular fluorine, and N-fluoropyridinium salts as represented by Umemoto reagent, but more preferred is a nitrogen- or argon-diluted perchloryl fluoride gas. Although the solvent used in the fluorination reaction of this synthetic procedure is not particularly limited, an etheric solvent such as diethyl ether and tetrahydrofuran is preferred. As a base, lithium bis(trimethylsilyl)amide, lithium diisopropylamide, 1,1,1,3,3,3-hexamethyldisilazane, sodium hydride, potassium bis(trimethylsilyl)amide, sodium bis(trimethylsilyl)amide, or the like is usable.

Preferred examples of the oxidizing agent used in this production method include ruthenium compounds such as ruthenium tetraoxide, and ruthenium dioxide, ruthenium trichloride, and the hydrates thereof used together with a co-oxidizing agent. Further, permanganates such as potassium permanganate, manganates such as manganese acetylacetonate, m-chloroperbenzoic acid and the like can also be used.

As the co-oxidizing agent, periodates such as sodium periodate, periodic acid, perchlorates such as sodium perchlorate, bromates such as potassium bromate, hypochlorites such as sodium hypochlorite, persulfates such as potassium persulfate, potassium ferricyanide, lead tetraacetate, peroxides such as hydrogen peroxide and tert-butylhydroperoxide, peracetic acid, m-chloroperbenzoic acid, high valent iodine compounds such as iodosyl benzene, amine-N-oxide, oxygen and the like can be used, but sodium periodate may be more preferably used as a strong co-oxidizing agent in the condition that the pH is adjusted with phosphates such as disodium hydrogen phosphate or carbonates such as sodium hydrogen carbonate to perform the reaction using ruthenium dioxide.

The deprotection of an amino group and the removal of a silyl group used in the synthetic procedure may be performed by a conventionally known method.

### Best Mode for Carrying out the Invention

Hereinafter, the present invention will be specifically described by way of examples, but the present invention is not intended to be limited to these examples.

### EXAMPLE 1

### Synthesis of 3′-fluoro-5′-hydroxythalidomide

To a solution of (5-hydroxy-2-oxopiperidin-3-yl)carbamic acid tert-butyl ester [Compound 1] (727 mg, diastereomer mixture of cis:trans=3:1) in dimethylformamide (10.5 mL) were added tert-butyldimethylchlorosilane (714 mg), and imidazole (645 mg). The mixture was stirred at room temperature for 14 hours and then concentrated under reduced pressure. The residue was purified by silica gel chromatography (ethyl acetate/hexane=2) to give a diastereomeric mixture (cis:trans=3:1) of [5-(tert-butyldimethylsilanyloxy)-2-oxopiperidin-3-yl]carbamic acid tert-butyl ester [Compound 2] as a colorless solid (1.034 g, 95%).

¹H-NMR (400MHz, CDCl₃) (major isomer) 0.08 (3H, s), 0.11 (3H, s), 0.90 (9H, s), 1.45 (9H, s), 1.90 (1H, ddd, J=1.7, 12.4, 12.8 Hz), 2.44 (1H, m), 3.24 (1H, dddd, J=1.7, 3.2, 3.2, 12.4 Hz), 3.48 (1H, ddd, J=1.8, 3.7, 12.4 Hz), 4.22 (1H, m), 4.34 (1H, m), 5.29 (1H, br s), 6.01 (1H, br s)

### m.p. 122-124° C.

Trifluoroacetic acid (0.25 mL) was added dropwise to a solution of compound 2 (145 mg) in dichloromethane (4 mL) at 0° C. The mixture was allowed to warm to room temperature and stirred for 8 hours. The mixture was concentrated under reduced pressure and the residual trifluoroacetic acid was removed as azeotrope with toluene. The residue was dissolved with toluene (3 mL) and then triethylamine (176 mg), phthalic anhydride (93 mg), and molecular sieves 3A (150 mg) were added to the solution. The mixture was heated at reflux for 6 hours and then cooled to room temperature. The insoluble materials were removed by filtration with celite and then washed with dichloromethane. The combined filtrate was concentrated under reduced pressure, and the residue was then purified by silica gel chromatography (ethyl acetate/hexane=2) to give a diastereomeric mixture (cis:trans=5:1) of 2-[5′-(tert-butyldimethylsilanyloxy)-2′-oxopiperidin-3′-yl]isoindole-1,3-dione [Compound 3] as a colorless solid (147 mg, 93%).

¹H-NMR (400MHz, CDCl₃) (major isomer) 0.12 (3H, s), 0.15 (3H, s), 2.12 (1H, dddd, J=2.4, 4.0, 6.0, 13.2 Hz), 2.59 (1H, ddd, J=2.0, 12.4, 13.2 Hz), 3.34 (1H, ddd, J=2.4, 5.6, 12.4 Hz), 3.69 (1H, dd, J=2.8, 12.4 Hz), 4.35 (1H, m), 5.24 (1H, dd, J=6.0, 12.4 Hz), 5.69 (1H, brs), 7.69-7.74 (2H, m), 7.83- 7.87 (2H, m)

### m.p. 215-232° C.

To a solution of compound 3 (750 mg) in tetrahydrofuran (20 mL) and dimethylformamide (0.5 mL) was added dropwise a 1.5 M solution of lithium bis(trimethylsilyl)amide in tetrahydrofuran (2.0 mL) at −78° C. under a nitrogen atmosphere. After stirring the mixture at −70° C. for 20 minutes, benzyl chloroformate (343 μL) was added dropwise to the mixture. The mixture was stirred at −70° C. for 2 hours, quenched with saturated ammonium chloride aqueous solution, and then extracted with chloroform (100 mL×2). The combined organic layer was washed with brine, dried with sodium sulfate, and then concentrated under reduced pressure. The residue was purified by silica gel chromatography (chloroform/hexane/ethyl acetate=5/4/1) to give a diastereomeric mixture (cis:trans=5:1) of 5′-(tert-butyldimethylsilanyloxy)-3′-(1,3-dioxo-1,3-dihydroisoindol-2-yl)-2′-oxopiperidine-1′-carboxylic acid benzyl ester [Compound 4] as a colorless solid (955 mg, 94%).

¹H-NMR (400MHz, CDCl₃) (major isomer) 0.10 (1H, s), 0.13 (3H, s), 0.91 (9H, s), 2.14 (1H, dddd, J=2.0, 4.0, 6.4, 12.8 Hz), 2.71 (1H, ddd, J=2.4, 12.8, 12.8 Hz), 3.87 (1H, dd, J=2.4, 13.6 Hz), 3.98 (1H, ddd, J=2.0, 3.2, 13.6 Hz), 4.38 (1H, m), 5.29 (2H, s), 5.36 (1H, dd, J=6.4, 12.8 Hz), 7.27-7.41 (5H, m), 7.71-7.75 (2H, m), 7.83-7.88 (2H, m)

### m.p. 135-137° C.

A 1.6 M solution of lithium bis(trimethylsilyl)amide in tetrahydrofuran (0.694 mL) was added dropwise to a solution of the compound 4 (513 mg) in tetrahydrofuran (10 mL) at −78° C. under a nitrogen atmosphere. After stirring the mixture at −30° C. for 1 hour, perchloryl fluoride gas, which is generated from stirring a solution of potassium perchlorate (1.5 g) in fluorosulfonic acid (4.4 mL) at 80° C. and diluted with nitrogen, was introduced into the mixture at −30° C. to −10° C. for 2.5 hours. The mixture was quenched with saturated ammonium chloride aqueous solution and then extracted with ethyl acetate (40 mL×3). The combined organic layer was washed with brine, dried with sodium sulfate, and then concentrated under reduced pressure. The residue was purified by silica gel chromatography (hexane/ethyl acetate=6) to give the less polar isomer [Compound 5] (282 mg, 53%) and the more polar isomer [Compound 6] (121 mg, 23%) of 5′-(tert-butyldimethylsilanyloxy)-3′-(1,3-dioxo-1,3-dihydroisoindol-2-yl)-3′-fluoro-2′-oxopiperidine-1′-carboxylic acid benzyl ester separately as a colorless solid. The less polar isomer [Compound 5] that fluorine atom at the 3′ position is syn to hydrogen at the 5′ position, represents syn isomer and the more polar isomer [Compound 6] that fluorine atom at the 3′ position is anti to hydrogen at the 5′ position, represents anti isomer.

### Compound 5

¹H-NMR (400MHz, CDCl₃) −0.06 (3H, s), 0.00 (3H, s), 0.68 (9H, s), 2.46 (1H, ddd, J=6.6, 8.1, 14.7 Hz), 3.26 (1H, dd, J=6.6, 13.7 Hz), 3.57 (1H, ddd, J=2.5, 10.3, 14.7 Hz), 4.38 (1H, m), 4.44 (1H, dd, J=7.3, 13.7 Hz), 5.37 (1H, d, J=12.8 Hz), 5.40 (1H, d, J=12.8 Hz), 7.31-7.40 (3H, m), 7.50-7.51 (2H, m) 7.77-7.81 (2H, m), 7.87-7.90 (2H, m)

### m.p. 135-137° C.

### Compound 6

¹H-NMR (400MHz, CDCl₃) 0.04 (3H, s), 0.07 (3H, s), 0.84 (9H, s), 2.35 (1H, ddd, J=6.4, 10.5, 14, 7 Hz), 3.54 (1H, dd, J=3.2, 14.0 Hz), 3.89 (1H, ddd, J=7.3, 8.2, 14.7 Hz), 4.18 (1H, m), 4.21 (1H, dd, J=3.2, 14.0 Hz), 5.39 (2H, s), 7.30-7.39 (3H, m), 7.49-7.51 (2H, m), 7.79-7.83 (2H, m), 7.89-7.92 (2H, m)

### m.p. 110° C.

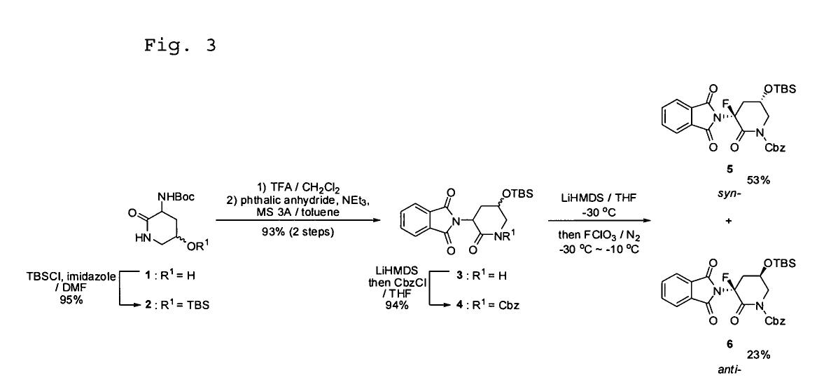

The synthesis of 3′-fluoro-5′-hydroxythalidomide is depicted in FIG. 3.

To a solution of compound 5 (319 mg) in ethanol/tetrahydrofuran=4/3 (7 mL) was added 10% palladium carbon (32 mg). The mixture was vigorously stirred at room temperature for 1 hour under a hydrogen atmosphere. The resulting mixture was filtered with cerite and the filtrate was concentrated under reduced pressure to give the syn isomer of 2-[5′-(tert-butyldimethylsilanyloxy)-3′-fluoro-2′-oxopiperidin-3′-yl]isoindole-1,3-dione [Compound 7] (268 mg, quant.) as a colorless solid. The crude product was used in the next reaction without purification.

1H-NMR (400MHz, CDCl3) −0.03 (3H, s), −0.01 (3H, s), 0.73 (9H, s), 2.59 (1H, ddd, J=4.4, 12.0, 14.4 Hz), 3.12 (1H, ddd, J=6.4, 14.4, 18.8 Hz), 3.26 (1H, m), 3.58 (1H, ddd, J=5.0, 5.5, 12.8 Hz), 4.37 (1H, dddd, J=2.8, 4.4, 6.4, 12.8 Hz), 6.21 (1H, br s), 7.73-7.78 (2H, m), 7.84-7.88 (2H, m)

### m.p. 164° C.

To a solution of the compound 7 (245 mg) in 1,2-dichloroethane (12 mL) were added H₂O (8 mL), sodium periodate (2.59 g), disodium hydrogen phosphate (1.72 g), and ruthenium dioxide hydrate (20 mg). The mixture was heated at reflux with vigorously stirring for 6.5 hours (the color of the solution became yellow). After a small amount of isopropanol was added to the mixture (black precipitates were formed in the reaction solution), the mixture was cooled. The insoluble materials were removed by filtration with cerite and the filtrate was extracted with chloroform (20 mL×3). The combined organic layer was washed with a mixture of brine (10 mL) and saturated sodium thiosulfate aqueous solution (5 mL), dried with sodium sulfate, and then concentrated under reduced pressure. The residue was purified by silica gel chromatography (ethyl acetate/hexane=½→2) to give the syn isomer of 2-[5′-(tert-butyldimethylsilanyloxy)-3′-fluoro-2′,6′-dioxopiperidin-3′-yl]isoindole-1,3-dione [Compound 9] (152 mg, 62%) as a colorless solid, together with the unreacted starting material (38 mg, 17%). ¹H-NMR (400MHz, CDCl₃) −0.06 (3H, s), 0.00 (3H, s), 0.61 (9H, s), 2.56 (1H, ddd, J=4.1, 7.8, 14.7 Hz), 3.64 (1H, ddd, J=4.6, 8.7, 14.7 Hz), 4.54 (1H, dd, 4.1, 8.7 Hz), 7.77-7.80 (2H, m), 7.87-7.90 (2H, m), 8.05 (1H, br s)

### m.p. 199-202° C.

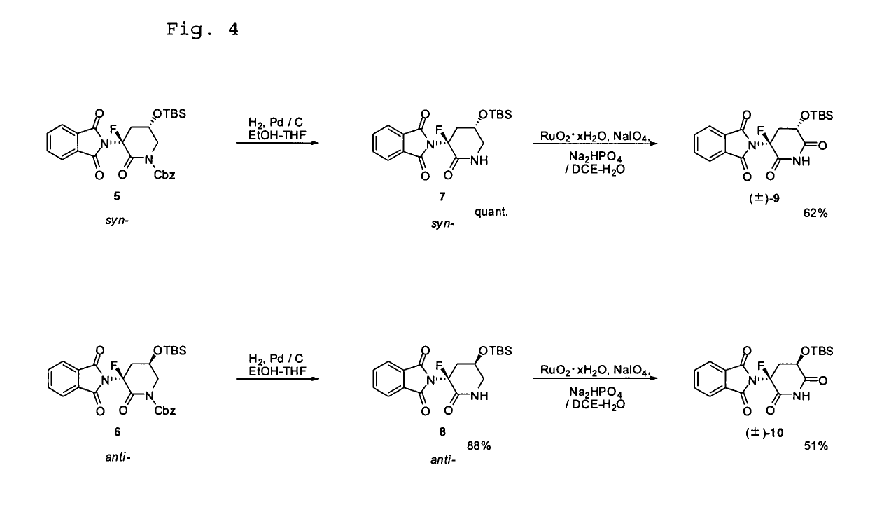

The synthesis of compound 7 and compound 9 from compound 5 is depicted in FIG. 4.

To a solution of compound 6 (161 mg) in ethanol/tetrahydrofuran=4/3 (3.5 mL) was added 10% palladium carbon (16 mg). The mixture was vigorously stirred at room temperature for 1 hour under a hydrogen atmosphere. The resulting mixture was filtered with cerite and the filtrate was concentrated under reduced pressure to give the anti isomer of 2-[5′-(tert-butyldimethylsilanyloxy)-3′-fluoro-2′-oxopiperidin-3′-yl]isoindole-1,3-dione [Compound 8] (105 mg, 88%) as a colorless solid. The crude product was used in the next reaction without purification.

¹H-NMR (400MHz, CDCl₃) 0.03 (3H, s), 0.06 (3H, s), 0.86 (9H, s), 2.51 (1H, ddd, J=8.4, 11.5, 14.0 Hz), 3.30 (1H, ddd, J=3.3, 5.6, 12.4 Hz), 3.36 (1H, ddd, J =4.8, 11.0, 14.0 Hz), 3.53 (1H, ddd, J=2.7, 4.4, 12.4 Hz), 4.17 (1H, dddd, J=4.4, 4.8, 5.6, 8.4 Hz), 6.06 (1H, brs), 7.77-7.80 (2H, m), 7.88-7.91 (2H, m)

### m.p. 198-200° C.

To a solution of the compound 8 (105 mg) in 1,2-dichloroethane (5.4 mL) were added H₂O (4 mL), sodium periodate (1.14 g), disodium hydrogen phosphate (760 mg), and ruthenium dioxide hydrate (18 mg). The mixture was heated at reflux with vigorously stirring for 4 hours (the color of the solution became yellow). After a small amount of isopropanol was added to the mixture (black precipitates were formed in the reaction solution), the mixture was cooled. The insoluble materials were removed by filtration with cerite and the filtrate was extracted with chloroform (15 mL×3). The combined organic layer was washed with a mixture of brine (10 mL) and saturated sodium thiosulfate aqueous solution (5 mL), dried with sodium sulfate, and then concentrated under reduced pressure. The residue was purified by silica gel chromatography (hexane/ether=5/2) to give the anti isomer of 2-[5′-(tert-butyldimethylsilanyloxy)-3′-fluoro-2′,6′-dioxopiperidin-3′-yl]isoindole-1,3-dione [Compound 10] (55.5 mg, 51%) as a colorless solid.

¹H-NMR (400MHz, CDCl₃) 0.08 (3H, s), 0.13 (3H, s), 0.89 (9H, s), 2.56 (1H, ddd, J=5.5, 12.4, 13.7 Hz), 3.88 (1H, ddd, J=3.5, 5.2, 13.7 Hz), 4.25 (1H, ddd, J=1.5, 5.2, 12.4 Hz), 7.83-7.89 (2H, m), 7.93-7.96 (2H, m), 8.24 (1H, brs)

### m.p. 212-215° C.

The synthesis of compound 8 and compound 10 from compound 6 is depicted in FIG. 4.

### Optical Resolution of Compound 9

The racemic mixture of compound 9 was separated by HPLC (DAICEL CHEMICAL INDUSTRIES, LTD., CHIRALPAK IA 1×25 cm, hexane/isopropanol=1/1, 0.75 mL/min), and then recrystallized from hexane and dichloromethane to give 2-[(3′S,5′S)-5′-(tert-butyldimethylsilanyloxy)-3′-fluoro-2′,6′-dioxopiperidin-3′-yl]isoindole-1,3-dione [Isomer 1] and 2-[(3′R,5′R)-5′-(tert-butyldimethylsilanyloxy)-3′-fluoro- 2′,6′-dioxopiperidin-3′-yl]isoindole-1,3-dione [Isomer 2] in optically pure form.
Isomer 1 (retention time: 66 min)
[α]_{D}−152.70°(c=0.1867, T=29.1, CHCl₃),
m.p. 201-204° C.
Isomer 2 (retention time: 92 min)
[α]_{D}+154.40 (c=0.2000, T=29.1, CHCl₃).
m.p. 202-205° C.

### Optical Resolution of Compound 10

The racemic mixture of compound 10 was separated by HPLC (DAICEL CHEMICAL INDUSTRIES, LTD., CHIRALPAK IA 1×25 cm, hexane/isopropanol=1/1, 0.75 mL/min), and then recrystallized from hexane and dichloromethane to give 2-[(3′R,5′S)-5′-(tert-butyldimethylsilanyloxy)-3′-fluoro-2′,6′-dioxopiperidin-3′-yl]isoindole-1,3-dione [Isomer 1] and 2-[(3′S,5′R)-5′-(tert-butyldimethylsilanyloxy)-3′-fluoro-2′,6′-dioxopiperidin-3′-yl]isoindole-1,3-dione [Isomer 2] in optically pure form.
Isomer 1 (retention time: 51 min)
[α]_{D}+109.80 (c=0.2000, T=28.7, CHCl₃)
m.p. 192-195° C.
Isomer 2 (retention time: 66 min)
[α]_{D}−110.3° (c=0.2000, T=28.9, CHCl₃)
m.p. 192-194° C.

Each stereoisomer of compounds 9 or 10 was applied to the following general procedure to obtain the corresponding stereoisomer of 3′-fluoro-5′-hydroxythalidomide.

### General Procedure

To a solution of compound 9 or 10 in acetonitrile (1 mL) was added dropwise 46% aqueous hydrofluoric acid (20 drops, about 0.15 mL) at 0° C. and the mixture was stirred at room temperature. After overnight (ca. 20 hours) stirring, the reaction mixture was diluted with ethyl acetate (3 mL) and washed with brine (2 mL). The aqueous layer was extracted with ethyl acetate (3 mL×2) and the combined organic layer was washed with saturated sodium hydrogen carbonate aqueous solution and brine, dried with sodium sulfate, and then concentrated under reduced pressure. The residue was purified by recrystallization from ethanol and hexane (the residue was subjected to silica gel chromatography before recrystallization, if necessary) to give optically pure compound 11 or 12, respectively, from compound 9 or 10.

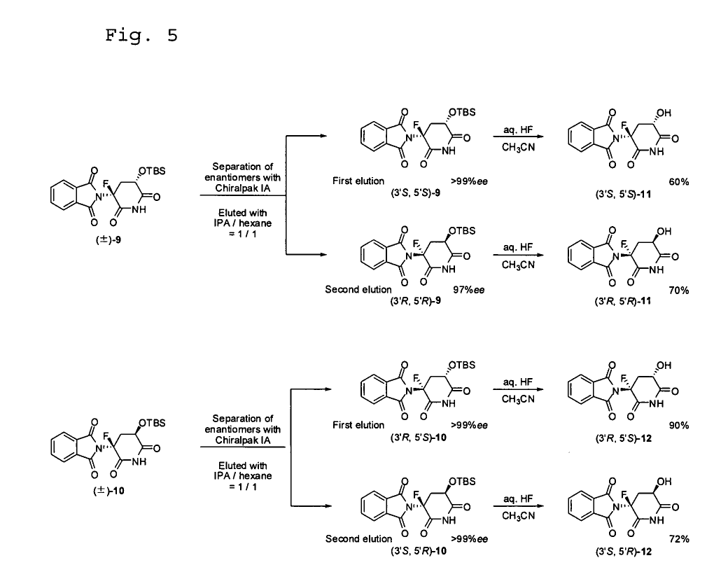

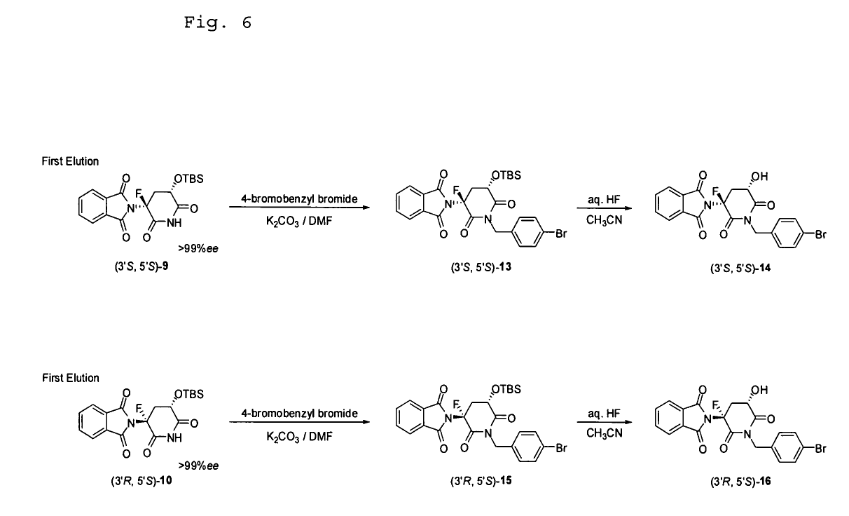

The optical resolution of compounds 9 and 10, and the synthesis of optically pure compounds 11 and 12, from compounds 9 and 10, respectively, are depicted in FIG. 5.

[2-(3′S,5′S)-3′-fluoro-5′-hydroxy-2′,6′-dioxopiperidin-3′-yl]isoindole-1,3-dione [(3′S,5′S)-11] [(from 10.2 mg of the corresponding stereoisomer of compound 9), 4.4 mg, 60%]
colorless solid
[α]_{D}−242.90 (c=0.1000, T=28.9, DMF)
¹H-NMR (400MHz, DMSO-d₆) 2.70 (1H, ddd, J=4.1, 7.8, 14.2 Hz), 3.28 (1H, ddd, J=4.6, 9.2, 14.2 Hz), 4.30 (1H, m), 6.24 (1H, d, J=5.5 Hz), 7.90-7.97 (4H, m), 11.65 (1H, br s)
   m.p. 250° C. (decomp.),

[2-(3′S, 5′S)-3′-fluoro-5′-hydroxy-2′,6′-dioxo-piperidin-3′-yl]isoindole-1,3-dione [(3′S, 5′S)-11] [(from 10.8 mg of the corresponding stereoisomer of compound 9), 5.4 mg, 70%]
colorless solid
[α]_{D}+204.9° (c=0.1067, T=28.8, DMF)
   ¹H-NMR (400MHz, DMSO-d₆) 2.70 (1H, ddd, J=4.1, 7.8, 14.2 Hz), 3.28 (1H, ddd, J=4.6, 9.2, 14.2 Hz), 4.30 (1H, m), 6.24 (1H, d, J=5.5 Hz), 7.90-7.97
(4H, m), 11.65 (1H, br s)
   m.p. 248° C. (decomp.)

[2-(3′R, 5′S)-3′-fuoro-5′-hydroxy-2′,6′-dioxopiperidin-3′-yl] isoindole-1,3-dione [(3′R, 5′S)-12] [(from 6.8 mg of the corresponding stereoisomer of compound 10), 4.4 mg, 90%]
colorless solid
[α]_{D}+204.00 (c=0.1000, T=28.9, DMF)
1H-NMR (400MHz, DMSO-d₆) 2.49 (1H, m), 3.51 (1H, ddd, J=2.3, 4.6, 12.4 Hz), 4.49 (1H, ddd, J=4.6, 6.0, 11.4 Hz), 5.94 (1H, d, J=6.0 Hz), 7.88-7.97 (4H, m), 11.64 (1H, s)
   m.p. 204 ° C (decomp.)

[2-(3′S, 5′R)-3′-fuoro-5′-hydroxy-2′,6′-dioxopiperidin-3′-yl]isoindole-1,3-dione [(3′S, 5′R)-12] [(from 8.1 mg of the corresponding stereoisomer of compound 10), 4.2 mg, 72%]
colorless solid
[α]_{D}−195.9° (c=0.1000, T=29.2, DMF)
1H-NMR (400MHz, DMSO-d₆) 2.49 (1H, m), 3.51 (1H, ddd, J=2.3, 4.6, 12.4 Hz), 4.49 (1H, ddd, J=4.6, 6.0, 11.4 Hz) 5.94 (1H, d, J=6.0 Hz), 7.88-7.97 (4H, m), 11.64 (1H, s)
   m.p. 203° C. (decomp.)

### EXAMPLE 2

### Determination of Absolute Configuration of 3′-fluoro-5′-hydroxythalidomide

To a solution of the first HPLC-eluted isomer of compound 9 (8.5 mg) in DMF (0.5 mL) were added 4-bromobenzyl bromide (6.3 mg) and potassium carbonate (3.2 mg) at 0° C. and the mixture was stirred at room temperature for 5 hours under a nitrogen atmosphere. A 1N aqueous solution of potassium hydrogen sulfate was added to the reaction mixture, and the resulting mixture was extracted with ethyl acetate. The organic layer was washed with brine, dried with sodium sulfate, and concentrated under reduced pressure. The residue was purified by silica gel chromatography (hexane/ethyl acetate=5) to give 2-[(3′S, 5′S)-1′-(4″-bromobenzyl)-3′-fluoro-5′-(isopropyldimethylsilanyloxy)-2′,6′-dioxopiperidin-3′-yl]isoindole-1,3-dione [Compound 13] (10.2 mg, 85%) as a colorless oil.

¹H-NMR (400MHz, CDCl₃) −0.09 (3H, s), −0.02 (3H, s), 0.59 (9H, s), 2.48 (1H, ddd, J=4.1, 7.3, 15.1 Hz), 3.63 (1H, ddd, J=3.2, 6.9, 15.1 Hz), 4.53 (1H, dd, J=4.1, 7.3 Hz), 5.03 (2H, s), 7.36 (2H, d, J=8.7 Hz), 7.45 (2H, d, J =8.7 H 7.77-7.79 (2H, m), 7.84-7.88 (2H, m)

A 46% aqueous hydrofluoric acid (0.1 mL) was added to a solution of compound 13 (10.2 mg) in acetonitrile (0.5 mL) at 0° C. and the mixture was stirred at room temperature for 3 hours. An additional 46% aqueous hydrofluoric acid (0.1 mL) was added to the mixture. After stirring at room temperature for 6 hours, the mixture was diluted with ethyl acetate (3 mL) and washed with brine (2 mL). The aqueous layer was extracted with ethyl acetate (3 mL×2), and the combined organic layer was washed with an aqueous saturated sodium hydrogen carbonate aqueous solution (1 mL) and brine, dried with sodium sulfate, and concentrated under reduced pressure. The residue was purified by silica gel chromatography (hexane/ethyl acetate=5) to give 2-[(3′S,5′S)-1′-(4″-bromobenzyl)-3′-fluoro-5′-hydroxy-2′, 6′-dioxopiperidin-3′-yl]isoindole-1,3-dione [Compound 14] (7.3 mg, 89%) as a colorless solid. Compound 14 was recrystallized from ether and hexane to give a crystal suitable for X-ray crystallographic analysis.

¹H-NMR (400MHz, CDCl₃) 2.92 (1H, ddd, J=6.0, 9.6, 14.5 Hz), 3.13 (1H, d, J=2.3 Hz), 3.25 (1H, ddd, J=8.4, 14.5, 24.8 Hz), 4.59 (1H, m), 4.97 (1H, d, J=14.0 Hz), 5.05, (1H, d, J=14.0 Hz), 7.30-7.33 (2H, m), 7.46-7.47 (2H, m), 7.78-7.81 (2H, m), 7.87-7.90 (2H, m)

### m.p. 159-161° C.

To a solution of the first HPLC-eluted isomer of compound 10 (9.2 mg) in DMF (0.5 mL) were added 4-bromobenzyl bromide (7.4 mg) and potassium carbonate (3.4 mg) at 0° C. and the mixture was stirred at room temperature for 4 hours under a nitrogen atmosphere. A 1N aqueous solution of potassium hydrogen sulfate was added to the reaction mixture, and the resulting mixture was extracted with ethyl acetate. The organic layer was washed with brine, dried with sodium sulfate, and concentrated under reduced pressure. The residue was purified by silica gel chromatography (hexane/ethyl acetate=4) to give 2-[(3′R,5′S)-1′-(4″-bromobenzyl)-3′-fluoro-5′-(isopropyldimethylsilanyloxy)-2′,6′-dioxopiperidin-3′-yl]isoindole-1,3-dione [Compound 15] (13.2 mg, quant) as a colorless oil.

1H-NMR (400MHz, CDCl₃) 0.06 (3H, s), 0.11 (3H, s), 0.86 (9H, s), 2.48 (1H, ddd, J=6.0, 11.9, 13.3 Hz), 3.78 (1H, ddd, J=4.1, 5.0, 13.3 Hz), 4.24 (1H, ddd, J=1.4, 5.0, 11.9 Hz), 4.93 (1H, d, J=14.0 Hz), 5.04 (1H, d, J=14.0 Hz), 7.37-7.39 (2H, m), 7.44-7.47 (2H, m), 7.82-7.87 (2H, m), 7.90-7.93 (2H, m)

A 46% aqueous hydrofluoric acid (0.2 mL) was added to a solution of compound 15 (10.2 mg) in acetonitrile (0.5 mL) at 0° C. and the mixture was stirred at room temperature for 2 hours. The mixture was diluted with ethyl acetate (3 mL) and washed with brine (2 mL). The aqueous layer was extracted with ethyl acetate (3 mL×2) and the combined organic layer was washed with saturated sodium hydrogen carbonate aqueous solution and brine, dried with sodium sulfate, and concentrated under reduced pressure. The residue was purified by silica gel chromatography (hexane/ethyl acetate=3) to give 2-[(3′R,5′S)-1′-(4″-bromobenzyl)-3′-fluoro-5′-hydroxy-2′, 6′-dioxopiperidin-3′-yl]isoindole-1,3-dione [Compound 16] (10.5 mg, quant.) as a colorless solid. Compound 16 was recrystallized from choloroform and ethanol to give a crystal suitable for X-ray crystallographic analysis.

1H-NMR (400MHz, CDCl₃) 2.42 (1H, ddd, J=6.4, 12.8, 13.3 Hz), 3.32 (1H, s), 3.85 (1H, ddd, J=1.4, 5.0, 13.3 Hz), 4.23 (1H, ddd, J=1.4, 5.0, 12.8 Hz), 4.98 (1H, d, J=13.7 Hz), 5.08 (1H, d, J=13.7 Hz), 7.36-7.41 (2H, m), 7.45-7.51 (2H, m), 7.80-7.86 (2H, m), 7.88-7.93 (2H, m)
m.p. 206° C. (decomp.)

Based on the X-ray crystal analysis of compound 14, the absolute configuration of (−)-11 and (+)-11 was determined unambiguously to be (3′S,5′S)-(−)-11 and (3′R,5′R)-(+)-11.

Based on the X-ray crystal analysis of compound 16, the absolute configuration of (−)-12 and (+)-12 was determined unambiguously to be (3′S,5′R)-(−)-12 and (3′R,5′S)-(+)-12.

The determination of absolute configuration of 3′-fluoro-5′-hydroxythalidomide of Example 2 is depicted in FIG. 6.

### Industrial Applicability

According to the present invention, all four stereoisomers of 3′-fluoro-5′-hydroxythalidomide can be obtained in optically pure form. As analogs of thalidomide, the 3′-fluoro-5′-hydroxythalidomide derivatives are useful in the treatment of erythema nodosum of Hansen's disease, prurigo, rheumatism, Crohn's disease, graft-versus-host disease, Behcet's disease, myeloma, aphtha ulcer and the like. Further, the 3′-fluoro-5′-hydroxythalidomide derivative is useful as a drug used for treatment of multiple myeloma, TNF-α related diseases and the like. Furthermore, the use of optically pure 3′-fluoro-5′-hydroxythalidomide derivative can be expected to be a novel drug having no teratogenesis, which is a side effect of thalidomide.

1. A 3′-fluoro-5′-hydroxythalidomide derivative represented by the general formula: 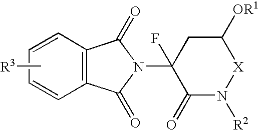
wherein R¹ represents a hydrogen atom, or an optionally substituted silyl, alkyl, cycloalkyl, aralkyl, aryl or acyl group; R² represents a hydrogen atom, or an optionally substituted hydroxyl, alkoxy, acyl, acyloxy or alkyl group; R³ represents, the same or different, 1 to 4 atoms or groups selected from a group consisting of hydrogen atom, a lower alkyl group, a lower alkoxy group, a halogen atom, a halogenated lower alkyl group, an amino group which may have a substituent, a lower alkylthio group, a lower alkoxycarbonyl group, a carbamoyl group which may have a substituent, a cyano group, a lower alkenyl group or a lower alkynyl group; and X represents CH₂ or C═O.

2. The 3′-fluoro-5′-hydroxythalidomide derivative according to claim 1, wherein R¹is a hydrogen atom or an optionally substituted silyl group; R2 is a hydrogen atom or an optionally substituted acyl group; and R3 is a hydrogen atom.

3. The 3′-fluoro-5′-hydroxythalidomide derivative according to claim 1, wherein R¹ is a hydrogen atom; R2 is a hydrogen atom; and R3 is a hydrogen atom.

4. The 3′-fluoro-5′-hydroxythalidomide derivative according to claim 1, which is an optically active (3′S, 5′R)-3′-fluoro-5′-hydroxythalidomide derivative, an optically active (3′R, 5′S)-3′-fluoro-5′-hydroxythalidomide derivative, an optically active (3′S, 5′S)-3′-fluoro-5′-hydroxythalidomide derivative, or an optically active (3′R, 5′R)-3′-fluoro-5′-hydroxythalidomide derivative.

5. The 3′-fluoro-5′-hydroxythalidomide derivative according to claim 1, wherein R¹ is a hydrogen atom; R² is a hydrogen atom; and R³ is a hydrogen atom, and
which is an optically active (3′S, 5′R)-3′-fluoro-5′-hydroxythalidomide derivative, an optically active (3′R, 5′S)-3′-fluoro-5′-hydroxythalidomide derivative, an optically active (3′S, 5′S)-3′-fluoro-5′-hydroxythalidomide derivative, or an optically active (3′R, 5′R)-3′-fluoro-5′-hydroxythalidomide derivative.
